(19) United States
(12) Patent Application Publication
(10) Pub. No.: US 2024/0000608 A1
(43) Pub. Date: Jan. 4, 2024

(54) NASOPHARYNGEAL AIRWAY MANAGEMENT DEVICE FOR BRAIN-NASAL CAVITY COOLING

(71) Applicant: AJOU UNIVERSITY INDUSTRY-ACADEMIC COOPERATION FOUNDATION, Suwon-si (KR)

(72) Inventor: Ji Man HONG, Yongin-si (KR)

(73) Assignee: AJOU UNIVERSITY INDUSTRY-ACADEMIC COOPERATION FOUNDATION, Suwon-si (KR)

(21) Appl. No.: 18/039,074

(22) PCT Filed: Nov. 16, 2021
(86) PCT No.: PCT/KR2021/016782

§ 371 Date: May 26, 2023

(30) Foreign Application Priority Data
Nov. 26, 2020 (KR) 10-2020-0161392
Nov. 16, 2021 (KR) 10-2021-0157350

(51) Int. Cl. A61F 7/12; F28F 1/00

(52) U.S. Cl. CPC A61F 7/12; F28F 1/00; A61F 2007/006

(57) ABSTRACT

The present invention provides a nasopharyngeal airway management device for brain-nasal cavity cooling according to the present invention including a cooling gas induction hose that is a tubular body with a hollow inside along a length, and induces a cooling gas introduced through one side to the other side; an adapter that is connected to the other side of the cooling gas induction hose, is mounted around the patient's upper lip, distributes the cooling gas induced through the cooling gas induction hose to a pair of discharge ripples, and discharges the cooling gas; and a nasal cavity cooling tube that is a tubular body with a hollow inside along a length, one side is coupled to a discharge nipple of the adapter and the other side of which is inserted into the patient's nasal cavity, and forms a plurality of discharge holes for discharging the cooling gas to any one point of the length, so that the nasal cavity cooling tube that is connected to the adapter fixed to the patient's upper lip, and dividing and discharging the cooling gas introduced through the cooling gas induction hose is inserted into the nasopharynx through the patient's nostril, and the cooling gas (low-temperature oxygen) discharged through the discharge hole of the the nasal cavity cooling tube is discharged into the nasal cavity close to the brain, and thereby quick and easy stable local cooling of the brain is induced while maintaining the nasopharyngeal airway.

### TECHNICAL FIELD

The present invention relates to a nasopharyngeal airway management device for brain-nasal cavity cooling, and particularly, to a nasopharyngeal airway management device for brain-nasal cavity cooling in which when a brain lesion (stroke, brain trauma, hypoxic encephalopathy, or the like) occurs, a nasal cavity cooling tube through which a cooling gas is discharged before significant brain tissue injury occurs is inserted into the nasal cavity, and the cooling gas is discharged through a discharge hole formed on a position corresponding to the nasal cavity in a length of the nasal cavity cooling tube, so that it is possible to quickly perform brain tissue therapeutic hypothermia with the cooling gas discharged through the nasal cavity cooling tube while maintaining the patient's airway.

### BACKGROUND

In general, compared to other tissues that may be regenerated, injury to the central nervous system is irreversible, so when brain injury occurs, death or many sequelae are displayed.

Such brain injury is temporally divided into primary injury and secondary injury due to various tissue cascades thereafter, depending on the causative mechanism of the primary injury.

Therapeutic hypothermia is one of the most powerful neuroprotective therapies that prevent irreversible secondary brain injury from occurring through various mechanisms after severe primary brain injury due to cardiac arrest, stroke, epilepsy, or the like.

Therapeutic hypothermia is known to be most effective in the mechanism of ischemia-reperfusion injury, and has been actively used as a major neuroprotective method, especially in a cardiac arrest situation.

However, therapeutic hypothermia to prevent brain injury may be divided into a method of lowering the temperature of the whole body and a method of lowering only the temperature of the brain, and for the method of lowering the temperature of the whole body, as the temperature decreases, chills, bradycardia, diuresis, hypokalemia, hyperglycemia, hemorrhage, and death may occur when the temperature drops below 30 degrees Celsius, so precise temperature control is essential.

For this reason, a temperature measuring device that accurately measures the patient's temperature and a complex cooling device (catheter or cooling pad) that enable accurate temperature control that works in conjunction with the temperature measuring device should be used, but excessive cooling that is not well-controlled is rather harmful to the patient.

On the other hand, since the method of lowering only the temperature of the brain may reduce the above-mentioned side effects of the method of lowering the temperature of the whole body, therapeutic cooling is possible even when the temperature is set at a temperature slightly lower than 33 degrees Celsius which is a temperature generally used in the whole body.

Selective brain-cooling is considered safe compared to whole body cooling because it may theoretically lower side effects on the whole body.

In fact, in a large-scale study of cardiac arrest published in The Journal of the American Medical Association (JAMA) in 2018, there were no more severe side effects on the whole body other than slightly more nosebleeds, and in the case of selective brain cooling, a good prognosis ratio of patients with a shockable rhythm tended to be higher.

So far, for selective brain-cooling, a cooling method using an artery going to the brain and a method using nasal cavity cooling have been used.

However, even when nasal cavity cooling is used, since an extracorporeal circulation device had to be operated, it is difficult to make the extracorporeal circulation device lightweight and difficult to manufacture the extracorporeal circulation device, and it was difficult for 119 members to carry the extracorporeal circulation device.

In addition, if the temperature of the refrigerant is too low and the refrigerant directly comes into contact with the skin or mucous membrane, the body may suffer from frostbite which may cause a safety problem.

As a conventional brain cooling technology, reference may be made to Korea Patent Registration No. 10-2023650 (Sep. 16, 2019).

### SUMMARY OF INVENTION

### Technical Problem

An object of the present invention is to provide a nasopharyngeal airway management device for brain-nasal cavity cooling in which a nasal cavity cooling tube that is connected to an adapter fixed to the patient's upper lip, and dividing and discharging a cooling gas introduced through a cooling gas induction hose is inserted into the nasopharynx through the patient's nostril, and the cooling gas (low-temperature oxygen) discharged through a discharge hole of the the nasal cavity cooling tube is discharged into the nasal cavity close to the brain, and thereby quick and easy stable local cooling of the brain is induced while maintaining the nasopharyngeal airway.

A nasopharyngeal airway management device for brain-nasal cavity cooling according to the present invention includes a cooling gas induction hose that is a tubular body with a hollow inside along a length, and induces a cooling gas introduced through one side to the other side; an adapter that is connected to the other side of the cooling gas induction hose, is mounted around the patient's upper lip, distributes the cooling gas induced through the cooling gas induction hose to a pair of discharge ripples, and discharges the cooling gas; and a nasal cavity cooling tube that is a tubular body with a hollow inside along a length, one side is coupled to a discharge nipple of the adapter and the other side of which is inserted into the patient's nasal cavity, and forms a plurality of discharge holes for discharging the cooling gas to any one point of the length.

In this case, the adapter according to the present invention includes a delivery chamber that has an inlet having one side connected to the other side of the cooling gas induction hose, and accommodates the cooling gas introduced through the cooling gas induction hose therein; a pair of discharge nipples that are formed at the front of the delivery chamber, and discharge the cooling gas accommodated in the delivery chamber; and a support that is provided on a lower side of the delivery chamber, and is seated around the patient's upper lip to support the delivery chamber.

In addition, the delivery chamber according to the present invention includes an insertion bar that has one side fixed to the inside of the discharge nipple and the other side inserted into the hollow of the nasal cavity cooling tube; and a sealing burr that is provided at an end of the insertion bar and seals the hollow of the nasal cavity cooling tube.

Here, the sealing burr according to the present invention opens up, down, left, or right in the hollow of the insertion bar to seal the hollow of the nasal cavity cooling tube.

In addition, it is preferable that the nasal cavity cooling tube according to the present invention forms a curved shape that is bent in any one direction.

In addition, it is preferable that the discharge holes of the nasal cavity cooling tube according to the present invention are formed on a position corresponding to the position of the nasal cavity close to the brain in the length of the nasal cavity cooling tube.

### Advantageous Effects

The effects of the nasopharyngeal airway management device for brain-nasal cavity cooling according to the present invention are as follows.

A nasal cavity cooling tube that is connected to an adapter fixed to the patient's upper lip, and dividing and discharging a cooling gas introduced through a cooling gas induction hose is inserted into the nasopharynx through the patient's nostril, and the cooling gas (low-temperature oxygen) discharged through a discharge hole of the the nasal cavity cooling tube is discharged into the nasal cavity close to the brain, and thereby quick and easy stable local cooling of the brain may be induced while maintaining the nasopharyngeal airway.

### BRIEF DESCRIPTION OF THE DRAWINGS

FIG. 1 is a reference view showing an example of use of a nasopharyngeal airway device for brain-nasal cavity cooling according to an embodiment of the present invention.

FIG. 2 is a reference view showing the nasopharyngeal airway device for brain-nasal cavity cooling according to an embodiment of the present invention.

FIG. 3 is a reference view showing a state in which a nasal cavity cooling tube is separated from an adapter according to an embodiment of the present invention.

FIG. 4 is a sectional view showing a cross section of the nasal cavity cooling tube according to an embodiment of the present invention.

FIG. 5 is a reference view showing an auxiliary bracket according to an embodiment of the present invention.

FIG. 6 is a reference view showing a state in which a nasal cavity cooling tube is separated from an adapter according to another embodiment of the present invention.

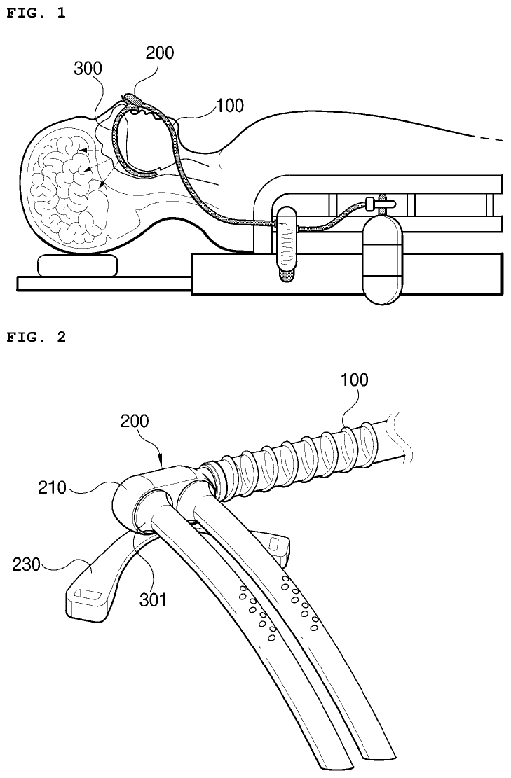

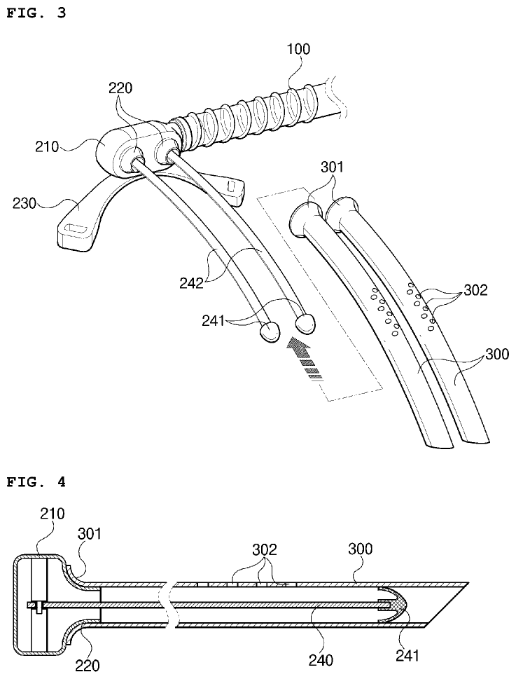

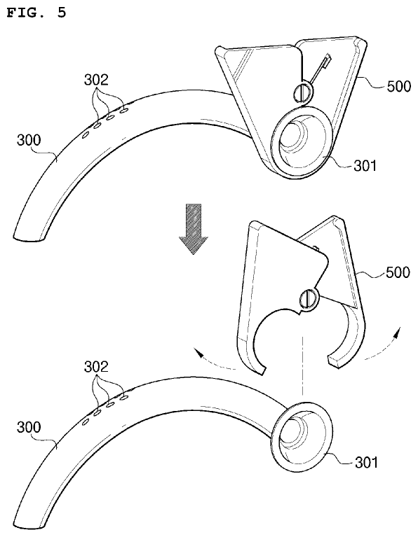

### BEST MODE FOR INVENTION

The present invention provides a nasopharyngeal airway management device for brain-nasal cavity cooling including a cooling gas induction hose that is a tubular body with a hollow inside along a length, and induces a cooling gas introduced through one side to the other side; an adapter that is connected to the other side of the cooling gas induction hose, is mounted around the patient's upper lip, distributes the cooling gas induced through the cooling gas induction hose to a pair of discharge ripples, and discharges the cooling gas; and a nasal cavity cooling tube that is a tubular body with a hollow inside along a length, one side is coupled to a discharge nipple of the adapter and the other side of which is inserted into the patient's nasal cavity, and forms a plurality of discharge holes for discharging the cooling gas to any one point of the length.

Hereinafter, preferred embodiments according to the present invention will be described in detail with reference to the accompanying drawings. Prior to this, the terms or words used in this specification and claims should not be construed as being limited to the usual or dictionary meaning, and should be interpreted as meanings and concepts consistent with the technical spirit of the present invention based on the principle that the inventor may appropriately define the concept of terms in order to explain his invention in the best way.

Therefore, the embodiments described in this specification and the configurations shown in the drawings are only the most preferred embodiments of the present invention, and do not represent all of the technical ideas of the present invention, so it should be understood that variations, which are equivalent alternatives that may be replaced at the time of the present application, may exist.

The present invention relates to a nasopharyngeal airway management device for brain-nasal cavity cooling in which when a brain lesion (stroke, brain trauma, hypoxic encephalopathy, or the like) occurs, the nasal cavity cooling tube through which a cooling gas is discharged before significant brain tissue injury occurs is inserted into the nasal cavity, and the cooling gas is discharged through a discharge hole formed on a position corresponding to the nasal cavity in a length of the nasal cavity cooling tube, so that it is possible to quickly perform brain tissue therapeutic hypothermia with the cooling gas discharged through the nasal cavity cooling tube while maintaining the patient's airway, and is described in more detail with reference to the drawings as follows.

The nasopharyngeal airway management device for brain-nasal cavity cooling according to an embodiment of the present invention includes a cooling gas induction hose 100, an adapter 200, and a nasal cavity cooling tube 300. First, the cooling gas induction hose 100 is a tubular body with a hollow inside along its length, and induces the cooling gas introduced through one side to the other side.

In this case, one side of the cooling gas induction hose 100 is connected to a cylinder containing the cooling gas, and the cooling gas discharged from the cylinder is introduced with the opening of the cylinder and guided from one side to the other side.

In addition, the cooling gas induction hose 100 is preferably provided with a coil on an outer circumferential surface in order to increase its durability.

The adapter 200 is connected to the other side of the cooling gas induction hose 100 and is mounted around the patient's upper lip so as to distribute and discharge the cooling gas induced through the cooling gas induction hose 100 to a pair of discharge ripples 220.

In this case, the adapter 200 includes a delivery chamber 210, the pair of discharge nipples 220, and a support 230. The delivery chamber 210 has an inlet 211 having one side connected to the other side of the cooling gas induction hose 100, and the cooling gas introduced through the cooling gas induction hose 100 is accommodated therein.

Here, the pair of discharge nipples 220 are formed at the front of the delivery chamber 210, and the pair of discharge nipples 220 discharge the cooling gas accommodated in the delivery chamber 210.

In addition, the support 230 is provided on the lower side of the delivery chamber 210, and the support 230 is seated around the patient's upper lip to support the delivery chamber 210.

In this case, the support 230 is preferably made of a rubber or silicone material having elasticity so as to come into close contact with the patient's upper lip, is formed in a shape corresponding to the patient's upper lip, and has both left and right ends coupled to an end of a strap or band. Therefore, the adapter 200 may be fixed to maintain a position around the patient's upper lip by wrapping the strap or band around the patient's head.

In addition, the delivery chamber 210 includes insertion bars 240, and the insertion bar 240 has one side fixed to the inside of the discharge nipple 220 and the other side inserted into the hollow of the nasal cavity cooling tube 300.

In this case, the insertion bar 240 is in a form of a thin bar having a thickness of 0.1 mm to 0.9 mm so as not to interfere with the flow of cooling gas in the hollow inside of the nasal cavity cooling tube 300 and is preferably made of stainless steel that does not corrode, and a plurality of perforations may be formed along the length thereof on a surface in order to reduce weight and secure fluidity of the cooling gas.

In addition, a sealing burr 241 is provided at an end of the insertion bar 240, and the sealing burr 241 seals the hollow of the nasal cavity cooling tube 300.

In this case, the sealing burr 241 is made of rubber or silicone material having elasticity, and is provided in an oblique line lying backward with respect to the entry direction up, down, left, or right, with the end of the insertion bar 240 at the center. The end of the insertion bar 240 provided with the sealing burr 241 forms an arrow (→) shape when viewed from a side or plane.

Therefore, when the insertion bar 240 is inserted into the hollow of the nasal cavity cooling tube 300, the sealing burr 241 lies down and enters the hollow of the nasal cavity cooling tube 300, and spreads toward the inner surface of the hollow of the nasal cavity cooling tube 300 due to elasticity when the insertion of the insertion bar is completed. Therefore, the sealing burr 241 comes into close contact with the inner surface of the hollow of the nasal cavity cooling tube 300, and closes the other side of the hollow of the nasal cavity cooling tube 300.

In addition, the sealing burr 241 is detachably coupled to the end of the insertion bar 240 to be selectively used, and a plurality of through-holes are formed in the sealing burr 241. Thus, by allowing some of the cooling gas flowing along the nasal cavity cooling tube 300 to escape, it is also possible to prevent the nasal cavity cooling tube 300 from being completely closed.

In addition, the nasal cavity cooling tube 300 is a tubular body with a hollow inside along the length, one side is coupled to the discharge nipple 220 of the adapter 200, the other side of which is inserted into the patient's nasal cavity, and forms a plurality of discharge holes 302 for discharging the cooling gas to any one point of the length.

In this case, the nasal cavity cooling tube 300 is formed with the hollow inside along the length like a conventional nasopharyngeal airway, and forms a curved shape bent to one side so as to be easily located in the nasopharynx through the nasal cavity.

In addition, the nasal cavity cooling tube 300 is made of silicone or rubber material that is harmless to the human body and has elasticity, so that the nasal cavity is not scratched or damaged when being inserted into the nose, and the tip of the nasal cavity cooling tube 300 forms a funnel-shaped expansion tube portion 301, so that when the nasal cavity cooling tube 300 is inserted into the nasal cavity, it is possible to limit the insertion depth.

In addition, the nasal cavity cooling tube 300 forms the discharge holes 302, and a plurality of the discharge holes 302 are preferably formed at positions corresponding to the nasal cavity in the length of the nasal cavity cooling tube 300.

Therefore, while the nasal cavity cooling tube 300 is inserted through the patient's nose and maintains the nasopharyngeal airway, the cooling gas (low-temperature oxygen) flowing through the hollow of the nasal cavity cooling tube 300 is discharged through the discharge hole 302, and the discharged cooling gas cools the nasopharynx, and thereby rapid, easy, and stable local cooling of the brain may be induced.

In addition, the expansion tube portion 301 may be formed as a tube connector so as to be connected to another tube connected to the ventilator.

Here, the nasopharyngeal airway device for brain-nasal cavity cooling according to an embodiment of the present invention may further include a vortex tube to inject cooling gas into the hollow of the cooling gas induction hose 100.

The vortex tube separates the compressed gas into a hot flow and a cold flow, may selectively provide only relatively cold gas, that is, cold air (low-temperature oxygen), and is connected to a portable cylinder containing high-pressure oxygen. Therefore, it is possible to generate relatively low-temperature oxygen using high-pressure oxygen emitted from the cylinder and provide cooling gas (low-temperature oxygen) into the hollow of the cooling gas induction hose 100.

Therefore, in the nasopharyngeal airway management device for brain-nasal cavity cooling according to an embodiment of the present invention, the vortex tube 400 is connected to the mobile oxygen cylinder provided in the firefighting vehicle, and by connecting the vortex tube and the cooling gas induction hose 100, the tube body 110 is located in the nasopharynx through the patient's nasal cavity, so that it is possible to transport the patient to the hospital while maintaining the airway during transport and performing local brain cooling together.

In addition, the nasal cavity cooling tube 300 is provided with an auxiliary bracket 500, the auxiliary bracket 500 has a ring-shaped tongs such as normal tongs formed on a lower side of a handle that is manipulated with the fingers, and a pair of tongs are rotatably coupled with a hinge axis while being symmetrically overlapped.

In this case, a hinge shaft is provided with a torsion spring, and the ring-shaped tongs are maintained in a faced state with each other due to the elasticity of the torsion spring.

Therefore, when the auxiliary bracket 500 presses the handle portion from the left and right with the hinge axis formed in the center as a pivot, the tongs open and when the pressing force applied in the left and right is released in a state in which the expansion tube portion 301 of the nasal cavity cooling tube 300 is accommodated in the widened tongs, the tongs face each other so that the auxiliary bracket 500 is fastened to the nasal cavity cooling tube 300.

The auxiliary bracket 500 may be used when the nasal cavity cooling tube 300 is coupled to or separated from the adapter 200.

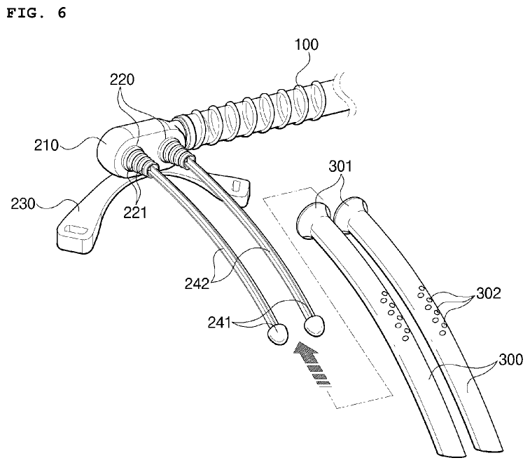

FIG. 6 shows a state in which the nasal cavity cooling tube is separated from the adapter according to another embodiment of the present invention. Referring to FIG. 6, the adapter 200 is connected to the other side of the cooling gas induction hose 100 and is mounted around the patient's upper lip, distributes the cooling gas induced through the cooling gas induction hose 100 to the pair of discharge ripples 220, and discharges the cooling gas.

In this case, the adapter 200 includes a delivery chamber 210, a pair of discharge nipples 220, and a support 230. The delivery chamber 210 has an inlet 211 having one side connected to the other side of the cooling gas induction hose 100, and accommodates the cooling gas introduced through the cooling gas induction hose 100 therein.

Here, the pair of discharge nipples 220 are formed at the front of the delivery chamber 210, and the pair of discharge nipples 220 discharge the cooling gas accommodated in the delivery chamber 210.

In this case, the discharge nipple 220 may be formed in multiple stages to be compatible with the nasal cavity cooling tubes 300 having various inner diameters.

Therefore, as the discharge nipples 220 are formed in multiple stages, the nasal cavity cooling tube 300 having an inner diameter corresponding to the size of the patient's nostrils may be used.

In addition, the delivery chamber 210 includes an insertion bar 240, the insertion bar 240 has one side fixed to the inside of the discharge nipple 220, the other side inserted into the hollow of the nasal cavity cooling tube 300.

In this case, the insertion bar 240 may have a vertical cross section formed in a ‘+’ shape to maintain rigidity without interfering the flow of the cooling gas in the hollow of the nasal cavity cooling tube 300.

Although the present invention has been described with reference to the embodiments shown in the drawings, these are only exemplary, and those skilled in the art will understand that various modifications and equivalent other embodiments are possible therefrom. Therefore, the true technical scope of protection of the present invention should be determined by the technical spirit of the appended claims.

1. A nasopharyngeal airway management device for brain-nasal cavity cooling comprising:
a cooling gas induction hose that is a tubular body with a hollow inside along a length, and induces a cooling gas introduced through one side to the other side;
an adapter that is connected to the other side of the cooling gas induction hose, is mounted around the patient's upper lip, distributes the cooling gas induced through the cooling gas induction hose to a pair of discharge ripples, and discharges the cooling gas; and
a nasal cavity cooling tube that is a tubular body with a hollow inside along a length, one side is coupled to a discharge nipple of the adapter and the other side of which is inserted into the patient's nasal cavity, and forms a plurality of discharge holes for discharging the cooling gas to any one point of the length.

2. The nasopharyngeal airway management device for brain-nasal cavity cooling of claim 1, wherein the adapter includes
a delivery chamber that has an inlet having one side connected to the other side of the cooling gas induction hose, and accommodates the cooling gas introduced through the cooling gas induction hose therein;
a pair of discharge nipples that are formed at the front of the delivery chamber, and discharge the cooling gas accommodated in the delivery chamber; and
a support that is provided on a lower side of the delivery chamber, and is seated around the patient's upper lip to support the delivery chamber.

3. The nasopharyngeal airway management device for brain-nasal cavity cooling of claim 2, wherein the delivery chamber includes
an insertion bar that has one side fixed to the inside of the discharge nipple and the other side inserted into the hollow of the nasal cavity cooling tube; and
a sealing burr that is provided at an end of the insertion bar and seals the hollow of the nasal cavity cooling tube.

4. The nasopharyngeal airway management device for brain-nasal cavity cooling of claim 3, wherein the sealing burr opens up, down, left, or right in the hollow of the insertion bar to seal the hollow of the nasal cavity cooling tube.

5. The nasopharyngeal airway management device for brain-nasal cavity cooling of claim 1, wherein the nasal cavity cooling tube forms a curved shape that is bent in any one direction.

6. The nasopharyngeal airway management device for brain-nasal cavity cooling of claim 1, wherein the discharge holes of the nasal cavity cooling tube are formed on a position corresponding to the position of the nasal cavity close to the brain in the length of the nasal cavity cooling tube.
